(19) United States
(12) Patent Application Publication
(10) Pub. No.: US 2024/0164401 A1
(43) Pub. Date: May 23, 2024

(54) AN ORAL NUTRACEUTICAL FORMULATION FOR RELIEVING SYMPTOMS OF MENSTRUATION

(71) Applicant: Deepak TOSHNIWAL, Pune (IN)

(72) Inventor: Deepak TOSHNIWAL, Pune (IN)

(21) Appl. No.: 18/280,852

(22) PCT Filed: Mar. 8, 2022
(86) PCT No.: PCT/IN2022/050208

§ 371 Date: Sep. 7, 2023

(30) Foreign Application Priority Data
Mar. 8, 2021 (IN) 202121009507

(51) Int. Cl. A23G 1/48; A23L 33/105; A23L 33/115; A61K 36/48; A61K 36/54; A61K 36/61; A61K 36/81; A61K 36/8965; A61K 36/906; A61K 36/9066

(52) U.S. Cl. CPC A23G 1/48; A23L 33/105; A23L 33/115; A61K 36/48; A61K 36/54; A61K 36/61; A61K 36/81; A61K 36/8965; A61K 36/906; A61K 36/9066

(57) ABSTRACT

The present invention relates to an oral nutraceutical formulation for relieving the symptoms of menstruation and a process of preparation of the formulation. The formulation consists of the ingredients dark chocolate and plant supplements including Ashwagandha powder, Shatavari powder, Dried Ginger powder, Ashok Sal powder, Nagkesar flowers, Turmeric, Cinnamon and Haritaki fruit. The formulation further includes Flaxseed oil, Sea salt and a combination of nuts and seeds. The formulation aims at relieving the symptoms of menstruation, Premenstrual syndrome (PMS) and Poly cystic ovary syndrome (PCOS) including bloating, mood swings, food craving along with relief from pain and cramps.

### FIELD OF THE INVENTION

The present invention relates to a nutraceutical formulation, and more particularly to an oral nutraceutical formulation for relieving symptoms of menstruation. The present invention also relates to a process for preparation of the oral nutraceutical formulation.

### BACKGROUND OF THE INVENTION

Menstruation refers to the monthly discharge of blood containing the lining of uterus. The process of menstruation also leads to fluctuation of the hormones. These physical and chemical changes in the body lead to various symptoms. The most common symptoms include pain, irritability, depression, mood swings, bloating, food cravings, oversensitivity and fatigue. The severity of the different symptoms also varies woman to woman depending on their everyday lifestyle and routine. The fluctuation of certain specific hormones (estrogen and cortisol) is believed to be directly related with the food cravings associated with menstruation. A neurotransmitter (Serotonin) associated with well-being influences the food intake and cravings during menstruation. The drop-in serotonin causes cravings for carbohydrates. The spike in cortisol levels and dip in serotonin levels increases cravings for sugary carbohydrates. A few ways for easing these symptoms include drinking plenty of fluids, eating a balanced diet, sleeping well to reduce fatigue. Also, analgesics are frequently used to relieve symptoms such as pain.

Various formulations are known in the art for relieving the pain caused due to menstruation. The Korean Patent Application KR100912912B1 by Kang Hong Gu discloses herbal supplements for treating menstrual pain. The Japanese Patent Application JP2011026217A by Yoshise Ran Emilie and others describe an oral menstrual pain-relieving agent containing an iron-bound type lactoferrin. The Chinese Patent Application CN106138586A by Nanjing Hospital of T.C.M disclose menstrual pain-relieving herbal formulation. The cited references disclose formulations only for relieving menstrual pain but no other symptoms of menstruation.

There is a need for a formulation for relieving the overall symptoms of menstruation including bloating, mood swings, food craving along with relief from pain and cramps. There is also a need of a formulation that not only alleviates the symptoms of menstruation but also provides nutrition to the body. Further there is a need for a formulation that is available in a form that is easily available and consumable in bite sizes portions. There is a further need for a process of preparing such a formulation.

### SUMMARY OF THE INVENTION

The present invention describes an oral nutraceutical formulation for relieving the symptoms of menstruation including bloating, mood swings, food craving along with relief from pain and cramps. The invention also aims at relieving the symptoms of Premenstrual syndrome (PMS) and Poly cystic ovary syndrome (PCOS). The oral nutraceutical composition comprises Dark Chocolate 500 gm; Plant extracts; Flaxseed Oil in the range of 20-25 ml; Nuts and seeds in the range of 150-200 gm and Sea salt in the range of 5-7 gm.

The plant extracts in the oral nutraceutical formulation include ashwagandha powder, shatavari powder, dried ginger powder, ashok sal powder, nagkesar flowers, turmeric, cinnamon and haritaki fruit. The dark chocolate used in the oral nutraceutical formulation is preferably sugar free dark chocolate obtained from cocoa powder. The nuts and seeds in the formulation are selected from Pecan, Almonds, Pistachio, Walnuts, Hazelnut, Pine Nut, Cashew, Chia seeds and Hemp seeds in the range 150-200 gm in equal proportion of each.

The formulation can optionally include additional flavoring agents selected from coffee, caramel, butterscotch and the like. The flavoring agents are added in the proportion of 5 ml flavoring agent per 500 gm of the formulation.

The oral nutraceutical formulation includes the ingredients in unit quantities as including Dark Chocolate: 500 gm; ashwagandha powder: 15-20 gm; shatavari powder: 15-17 gm; dried ginger powder: 15-20 gm; ashok sal powder: 15-17 gm; nagkesar flowers: 10-12 gm; turmeric: 10-15 gm; cinnamon: 10-12 gm; haritaki fruit: 10-15 gm; flaxseed oil: 20-25 ml; nuts and seeds: 150-200 gm; and sea salt: 5-7 gm.

Preferably, the oral nutraceutical formulation includes the ingredients in specific quantities as follows: sugar free dark chocolate: 500 gm; ashwagandha powder: 15 gm; shatavari powder: 15 gm; dried ginger powder: 15 gm; ashok sal powder: 15 gm; nagkesar flowers: 10 gm; turmeric: 10 gm; cinnamon: 10 gm; haritaki fruit: 15 gm; flaxseed oil: 20 ml; nuts and seeds: 200 gm; and sea salt: 5 gm.

The process of preparation of the oral nutraceutical formulation includes addition of a predefined quantity of dark chocolate in a heating equipment; treatment of the chocolate of the previous step with hot treatment including the steps of melting, tempering and homogenization taking place at a predefined temperature for a predefined period of time; treatment of the chocolate of the previous step by cold treatment at a predefined temperature for a predefined period of time; addition of a predefined quantity of the remaining ingredients to the cocoa paste and stirring under heat; optionally addition of predefined flavors to the mixture of previous step and mixing the formulation mixture and transferring to different moulds followed by cooling the mixture to form the final oral nutraceutical formulation. In this process, the hot treatment of the dark chocolate is carried out at 30° C. to 48° C. for 1.5 to 2 hours; and the cold treatment of the dark chocolate is carried out at 5° C. to 15° C. for 3 to 4 hours.

### DETAILED DESCRIPTION OF THE INVENTION

The foregoing objects of the present invention are accomplished and the problems and shortcomings associated with the prior art, techniques and approaches are overcome by the present invention as described below in the preferred embodiments.

All materials used herein were commercially purchased as described herein or prepared from commercially purchased materials as described herein.

Although specific terms are used in the following description for sake of clarity, these terms are intended to refer only to particular structure of the invention selected for illustration in the drawings and are not intended to define or limit the scope of the invention.

References in the specification to “preferred embodiment” means that a particular feature, structure, characteristic, or function described in detail thereby omitting known constructions and functions for clear description of the present invention.

In one aspect, the present invention relates to an oral nutraceutical formulation for relieving the symptoms of menstruation including bloating, mood swings, food craving along with relief from pain and cramps. The invention also aims at relieving the symptoms of Premenstrual syndrome (PMS) including mood swings, food cravings, fatigue, irritability and depression. It aims at relieving the symptoms of Poly cystic ovary syndrome (PCOS) including acne and menstrual irregularity.

In another aspect, the present invention provides a process for the preparation of the oral nutraceutical formulation.

The oral nutraceutical formulation comprises:
1. Dark Chocolate 500 gm;
2. Plant extracts;
3. Flaxseed Oil in the range of 20-25 ml;
4. Nuts and seeds in the range of 150-200 gm; and
5. Sea salt in the range of 5-7 gm

In accordance with a preferred embodiment, the plant extracts include ashwagandha powder, shatavari powder, dried ginger powder, ashok sal powder, nagkesar flowers, turmeric, cinnamon and haritaki fruit.

The nuts and seeds are selected from pecan, almonds, pistachio, walnuts, hazelnut, pine nut, cashew, chia seeds and hemp seeds in the range of 150-200 gm in equal proportion of each. In another embodiment, the nuts and seeds are selected from pecan, almonds, pistachio, walnuts, hazelnut, pine nut, cashew, chia seeds and hemp seeds in the range of 150-200 gm in random proportion.

The dark chocolate used is sugar free dark chocolate obtained from cocoa powder. The sugar free dark chocolate in accordance with the present invention is preferably defined by making a composition of dark cocoa powder, cocoa butter and maltitol. Accordingly, for obtaining a quantity of 100 gm sugar free dark chocolate, 80 gm cocoa powder, 20 gm of maltitol and 30 gm of cocoa butter is used.

The formulation is packaged in bite size portions to be consumed during the 4 to 5 days of menstruation.

In a further embodiment, the oral nutraceutical formulation includes the ingredients in unit quantities as below:

| | | |
|---|---|---|
| Sr. No. | INGREDIENTS | QUANTITY |

| 1. | | Dark Chocolate | 500 | gm |
|---|---|---|---|---|
| 2. | | Plant Supplements | | |
| | a) | Ashwagandha powder | 15-20 | gm |
| | b) | Shatavari powder | 15-17 | gm |
| | c) | Dried Ginger powder | 15-20 | gm |
| | d) | Ashok Sal powder | 15-17 | gm |
| | e) | Nagkesar flowers | 10-12 | gm |
| | f) | Turmeric | 10-15 | gm |
| | g) | Cinnamon | 10-12 | gm |
| | h) | Haritaki fruit | 10-15 | gm |

| | | | |
|---|---|---|---|
| 3. | Flaxseed Oil | 20-25 | ml |
| 4. | Nuts and Seeds | 150-200 | gm |
| 5. | Sea Salt | 5-7 | gm |

Further, the oral nutraceutical formulation optionally includes additional flavoring agents selected from coffee, caramel, butterscotch and the like. The flavoring agents are added in the proportion of 1 ml of flavoring agent per 100 gm of the formulation.

The formulation of the present invention helps in dealing with the symptoms of menstruation in many different ways. The dark sugar free chocolate and turmeric help in controlling mood swings. Dark chocolate stimulates the synthesis of serotonin which leads to overall increase in happiness. The flaxseed oil that is rich in Omega 3, Omega 6 fatty acids and the minerals from sea salt relax muscles, improve blood circulation and reduce bloating. The ingredients such as nuts and seeds rich in multi-vitamins (B1, B2 and B3) along with Ashwagandha act as fatigue improvisers and energy boosters. The ingredients Shatavari, Haritaki, Cinnamon, Nagkeshar help in soothing cramps. The nuts and seeds rich in vitamins (Vitamin C and Vitamin B12) and Iron along with ginger reduce bloating. The Vitamin C present in nuts and seeds, Cocoa and Cocoa Butter present in the dark chocolate along with other ingredients such as Haritaki and turmeric act as antioxidants. The Nagkesar flower helps in digestion during the periods.

Now a preferred process for the preparation of the oral nutraceutical formulation in accordance with the present invention is described. Accordingly, the process of preparation of the oral nutraceutical formulation includes the following steps:
a) The first step is adding a predefined quantity of dark sugar-free chocolate having cocoa, cocoa butter and maltitol in a heating equipment. In this step, the quantity of dark sugar free chocolate is, for example, 500 gm. The sugar free dark chocolate in accordance with the present invention is preferably defined by making a composition of dark cocoa powder, cocoa butter and maltitol. Accordingly, for obtaining a quantity of 100 gm sugar free dark chocolate 80 gm cocoa powder, 20 gm of maltitol and 30 gm of cocoa butter is used.
b) The next step includes treating the dark chocolate of above step with hot treatment. The hot treatment of dark chocolate includes the steps of melting, tempering and homogenization taking place at a predefined temperature for a predefined period of time. In this step, the hot treatment of the dark chocolate is carried out at a predefined temperature range of 30° C. to 48° C. for a predefined time period of 1.5 to 2 hours.
c) The next step includes treating the chocolate of previous step by cold treatment at a predefined temperature for a predefined period of time. The cold treatment of the chocolate is carried out at a predefined temperature range of 5° C. to 15° C. for a predefined time period of 3 to 4 hours.
d) The next step includes adding a predefined quantity of the remaining ingredients to the cocoa paste and stirring under heat. The ingredients are added preferably in the quantity including ashwagandha powder: 15 gm; shatavari powder: 15 gm; dried ginger powder: 15 gm; ashok sal powder: 15 gm; nagkesar flowers: 10 gm; turmeric: 10 gm; cinnamon: 10 gm; haritaki fruit: 15 gm; flaxseed oil: 20 ml; nuts and seeds: 200 gm; and sea salt: 5 gm.
e) The next step includes optionally adding predefined flavors to the mixture of step d). The flavoring agents are selected from coffee, caramel, butterscotch and the like. The flavoring agents are added in the proportion of 1 ml flavoring agent per 100 gm of the formulation.
f) The last step includes mixing the formulation mixture and transferring to different moulds followed by cooling the mixture to form the final formulation.

In this embodiment, the tempering of the cocoa powder during the hot treatment converts the powder to a semi-solid form. The hot treatment of the dark chocolate is carried out at a predefined temperature range of 30° C. to 48° C. for a predefined time period of 1.5 to 2 hours. The cold treatment of the dark chocolate is carried out at a predefined temperature range of 5° C. to 15° C. for a predefined time period of 3 to 4 hours.

The oral nutraceutical formulation of the present invention provides nutrition along with alleviating various symptoms of menstruation. The dark chocolate used is sugar-free in nature and does not add up to the calorie count of the consumer.

Advantageously, the oral nutraceutical formulation in bite size portions is a healthy and affordable snack for women helping them satisfy their food cravings during pre-menstrual syndrome (PMS) and menstruation.

### EXAMPLES

Only a few examples and implementations are disclosed. Variations, modifications, and enhancements to the described examples and implementations and other implementations can be made based on what is disclosed.

Examples are set forth herein below and are illustrative of different amounts and types of reactants and reaction conditions that can be utilized in practicing the disclosure. It will be apparent, however, that the disclosure can be practiced with other amounts and types of reactants and reaction conditions than those used in the examples, and the resulting devices various different properties and uses in accordance with the disclosure above and as pointed out hereinafter.

### Example 1: The Oral Nutraceutical Formulation Includes the Ingredients in Quantities as Specified in the Table Below

| | | |
|---|---|---|
| Sr. No. | INGREDIENTS | QUANTITY |

| 1. | | Dark Chocolate - Sugar Free | 500 | gm |
|---|---|---|---|---|
| 2. | | Plant Supplements | | |
| | a) | Ashwagandha | 15 | gm |
| | b) | Shatavari powder | 15 | gm |
| | c) | Dried Ginger powder | 15 | gm |
| | d) | Ashok Sal powder | 15 | gm |
| | e) | Nagkesar flowers | 10 | gm |
| | f) | Turmeric | 10 | gm |
| | g) | Cinnamon | 10 | gm |
| | h) | Haritaki fruit | 15 | gm |

| | | | |
|---|---|---|---|
| 3. | Flaxseed Oil | 20 | ml |
| 4. | Nuts and Seeds | 200 | gm |
| 5. | Sea Salt | 5 | gm |

### Example 2: The Process of Preparation of the Oral Nutraceutical Formulation is Illustrated as Follows

The formulation is prepared by adding 500 gm of dark sugar-free chocolate in a heating equipment. This is followed by treating the chocolate with hot treatment including the steps of melting, tempering and homogenization taking place at 40° C. for 2 hours. The tempering of the cocoa powder during the hot treatment converts the powder to a semi-solid form. Further the chocolate is treated by cold treatment at 10° C. for 3 hours. The plant supplements including 15 gm of ashwagandha powder, 15 gm of shatavari powder, 15 gm of dried ginger powder, 15 gm of ashok sal powder, 10 gm of nagkesar flowers, 10 gm of turmeric, 10 gm of cinnamon, 15 gm of haritaki fruit; and 20 ml of flaxseed oil, 200 gm of nuts and seeds, 5 gm of sea salt are added to the cocoa paste and stirred under heat. Optionally, a predefined flavor selected from coffee, caramel, butterscotch is added to the mixture of the previous step. The formulation mixture is mixed and transferred to different moulds followed by cooling the mixture to form the bite size portions of the formulation.

### Example 3: Nutritional Details

The nutritional value of the oral nutraceutical formulation per 100 grams is given as follows:

NUTRITIONAL VALUE PER 100 GMS

| | Parameters | Unit | Value |
|---|---|---|---|
| 1. | Energy | Kcal | 555.5 |
| 2. | Protein | gm/100 grams | 6.86 |
| 3. | Carbohydrates | gm/100 grams | 50.38 |
| 4. | Omega 3 | mg/100 grams | 30 |
| 5. | Omega 6 | mg/100 grams | 1210 |
| 6. | Vitamin B1 | mg/100 grams | 1.1 |
| 7. | Vitamin B2 | mg/100 grams | 1.7 |
| 8. | Vitamin B6 | mg/100 grams | 2.3 |

Vitamin C, B3, Magnesium, Iron, Calcium, Minerals

The embodiments were chosen and described in order to best explain the principles of the present invention and its practical application, to thereby enable others, skilled in the art to best utilize the present invention and various embodiments with various modifications as are suited to the particular use contemplated.

It is understood that various omission and substitutions of equivalents are contemplated as circumstance may suggest or render expedient, but such are intended to cover the application or implementation without departing from the spirit or scope of the present invention.

1. An oral nutraceutical formulation for relieving symptoms for PMS and PCOS relating to menstruation comprising:
a) Dark Chocolate 500 gm;
b) Plant extracts;
c) Flaxseed Oil in a range of 20-25 ml;
d) Nuts and seeds in a range of 150-200 gm; and
e) Sea salt in a range of 5-7 gm.

2. The oral nutraceutical formulation as claimed in claim 1, wherein the plant extracts include ashwagandha powder, shatavari powder, dried ginger powder, ashok sal powder, nagkesar flowers, turmeric, cinnamon and haritaki fruit.

3. The oral nutraceutical formulation as claimed in claim 1, wherein the dark chocolate is a sugar free dark chocolate obtained from cocoa powder.

4. The oral nutraceutical formulation as claimed in claim 1, wherein the nuts and seeds are selected from a group of pecan, almonds, pistachio, walnuts, hazelnut, pine nut, cashew, chia seeds and hemp seeds in the range of 150-200 gm in equal proportion of each.

5. The oral nutraceutical formulation of claim 1, that optionally includes flavoring agents selected from coffee, caramel, butterscotch and the like.

6. The oral nutraceutical formulation as claimed in claims 1 and 2, wherein the formulation includes the ingredients in unit quantities as follows:
a) Dark Chocolate: 500 gm;
b) Ashwagandha powder: 15-20 gm;
c) Shatavari powder: 15-17 gm;
d) Dried Ginger powder: 15-20 gm;
e) Ashok Sal powder: 15-17 gm;
f) Nagkesar flowers: 10-12 gm;
g) Turmeric: 10-15 gm;
h) Cinnamon: 10-12 gm;
i) Haritaki fruit: 10-15 gm;
j) Flaxseed Oil: 20-25 ml;
k) Nuts and Seeds: 150-200 gm; and
l) Sea Salt: 5-7 gm.

7. The oral nutraceutical formulation as claimed in claims 3 and 6, wherein the formulation includes the ingredients in unit quantities as follows:
a) Sugar free Dark Chocolate: 500 gm;
b) Ashwagandha powder: 15 gm;
c) Shatavari powder: 15 gm;
d) Dried Ginger powder: 15 gm;
e) Ashok Sal powder: 15 gm;
f) Nagkesar flowers: 10 gm;
g) Turmeric: 10 gm;
h) Cinnamon: 10 gm
i) Haritaki fruit: 15 gm;
j) Flaxseed Oil: 20 ml;
k) Nuts and Seeds: 200 gm; and
l) Sea Salt: 5 gm.

8. The process of preparation of the oral nutraceutical formulation as claimed in claim 1 including the steps of:
a) addition of a predefined quantity of dark chocolate in a heating equipment;
b) treatment of the chocolate of the previous step with hot treatment including the steps of melting, tempering and homogenization taking place at a predefined temperature for a predefined period of time;
c) treatment of the chocolate of the previous step by cold treatment at a predefined temperature for a predefined period of time;
d) addition of a predefined quantity of the remaining ingredients to the cocoa paste and stirring under heat;
e) optionally addition of predefined flavors to the mixture of previous step d); and
f) mixing the formulation mixture and transferring to different moulds followed by cooling the mixture to form the final formulation.

9. The process as claimed in claim 8, wherein the hot treatment of the dark chocolate is carried out at 30° C. to 48° C. for 1.5 to 2 hours.

10. The process as claimed in claim 8, wherein the cold treatment of the chocolate is carried out at 5° C. to 15° C. for 3 to 4 hours.
